SEQUENCE LISTING (4 sequences)

SEQ ID NO: 1 - DNA, 16 bp, Artificial Sequence
  REST transcription factor
ntgnncanggtgctga

SEQ ID NO: 2 - DNA, 14 bp, Artificial Sequence
  2NF423 transcription factor
gaaccctgcgggtc

SEQ ID NO: 3 - DNA, 14 bp, Artificial Sequence
  HAND1 transcription factor
ccagaccgcagaaa

SEQ ID NO: 4 - DNA, 10 bp, Artificial Sequence
  NEUROD1 transcription factor
cagattgcta

(19) United States
(12) Patent Application Publication
(10) Pub. No.: US 2014/0271455 A1
(43) Pub. Date: Sep. 18, 2014

(54) DNA METHYLATION BIOMARKERS FOR SMALL CELL LUNG CANCER

(71) Applicant: CITY OF HOPE, Duarte, CA (US)

(72) Inventors: Gerd P. PFEIFER, Bradbury, CA (US); Satish KALARI, Arcadia, CA (US); Marc JUNG, Duarte, CA (US)

(21) Appl. No.: 14/206,718

(22) Filed: Mar. 12, 2014

Related U.S. Application Data
(60) Provisional application No. 61/784,936, filed on Mar. 14, 2013.

(51) Int. Cl. C12Q 1/68

(52) U.S. Cl. CPC C12Q 1/6886
USPC 424/1.11; 435/6.11; 506/9; 506/2

(57) ABSTRACT

The methods provided herein relate to the identification of novel DNA biomarkers and the use of the aberrant methylation patterns of the DNA biomarkers to diagnose small cell lung cancer (SCLC). Such methods may include diagnosing SCLC when there is an increase in methylation of one or more DNA biomarkers in a test sample compared with that in a normal sample. DNA methylation patterns of DNA biomarkers on a genome-wide scale may be determined using a variety of methods including the methylated-CpG island recovery assay (MIRA). In some embodiments, methods of treating a subject for SCLC or monitoring the treatment are also provided. Methods may include measuring the methylation levels of one or a combination of DNA biomarkers and administering chemotherapy to a subject when there is an increase in the methylation levels of the test sample in relation to that of the normal sample or standard sample.

### PRIORITY CLAIM

The present application claims priority to U.S. Provisional Application No. 61/784,936, filed Mar. 14, 2013, which is incorporated by reference herein in its entirety, including the drawings.

### STATEMENT OF GOVERNMENT INTEREST

The present invention was made with government support under Grant No. CA084469 awarded by the National Institutes of Health/National Cancer Institute (NIH/NCl). The Government has certain rights in the invention.

### BACKGROUND

Lung cancer is divided by histology into small cell lung cancer (SCLC) and non-small cell lung cancer (NSCLC). SCLC represents about 15% of all lung cancer cases and is one of the most lethal forms of cancer with properties of high mitotic rate and early metastasis (Govindan et al., 2006). It is distinctly characterized by small cells with poorly defined cell borders and minimal cytoplasm, rare nucleoli and finely granular chromatin. Although SCLC patients initially respond to chemotherapy and radiation therapy, the disease recurs in the majority of patients. Because of the aggressiveness of SCLC and the lack of effective therapy and early diagnosis, without treatment the median survival time for SCLC is only 2-4 months. With current treatment modalities, the median survival times for limited-stage disease, <5% of the total, is 16-24 months and for extensive disease, 7-12 months, in spite of the fact that 60-80% of patients respond to therapy. It is essential to gain a better understanding of the molecular pathogenesis of the disease and to identify molecular alterations, which could lead to improved results in early detection and a means of assessing response to therapy.

However, epigenetic aberrations, specifically DNA methylation changes found in SCLC tumors, have not been studied so far in a comprehensive manner. Thus, there is a need to identify methylated regions that can provide specificity in discriminating SCLC tumors from normal tissue. As described herein, the methylated-CpG island recovery assay (MIRA), may be utilized to map DNA methylation patterns at promoters and CpG islands of primary SCLC tumors, SCLC cell lines and normal lung control samples. These novel methylation patterns may serve as DNA biomarkers for early detection and therapeutic management of SCLC.

### SUMMARY

One aspect of the invention relates to a method of diagnosing, determining or predicting that a subject has small cell lung cancer (SCLC) that is associated with aberrant methylation of DNA in a sample provided, wherein the method by measuring methylation levels of one or a combination of DNA biomarkers in a test sample from a subject, and comparing the methylation levels of the one or the combination of DNA biomarkers with the methylation levels of a corresponding one or a combination of DNA biomarkers in a normal sample or standard sample, and predicting that an increase in the methylation levels of the test sample in relation to that of the normal sample or standard sample indicates that the subject is likely to have SCLC. The aberrant methylation as referred to herein is hypermethylation. In some embodiments, the test sample may be obtained from lung tissue, bronchial biopsies, sputum, and/or blood serum.

The methylation of DNA often occurs at genome regions known as CpG islands. The CpG islands are susceptible to aberrant methylation (e.g., hypermethylation) in a stage- and tissue-specific manner during the development of a condition or disease (e.g., cancer). Thus the measurement of the level of methylation indicates the likelihood or the stage (e.g., onset, development, or remission stage) of SCLC.

The methylation of DNA can be detected via methods known in the art. In a preferred embodiment, the level can be measured via a methylated-CpG island recovery assay (MIRA), bisulfite sequencing, combined bisulfite-restriction analysis (COBRA) or methylation-specific PCR (MSP). In another preferred embodiment, the methylation levels of a plurality DNA can be measured through MIRA-assisted DNA array.

The DNA biomarkers are fragments of genome DNA which contain a CpG island or CpG islands, or alternatively, are susceptible to aberrant methylation. Examples of the DNA biomarkers associated with SCLC are disclosed in FIG. 5 and Table 6. Further examples of DNA biomarkers include GALNTL1, MIR-10A, MIR-129-2, MIR-196A2, MIR-615, MIR-9-3, AMBRA1, HOXD10, PROX1, ZNF672, DMRTA2, EOMES/TBR2, TAC1 and RESP18. The methylation of these biomarkers may occur at a frequency of greater than about 77% of primary SCLC tumors. In some embodiments, the methylation of these biomarkers may occur at a frequency of greater than about 33% of primary SCLC tumors.

Additionally, the DNA biomarkers may comprise a sequence motif that is identified in regions that are specifically methylated in SCLC tumor samples. These sequence motifs may be highly enriched compared with non-tumor specifically methylated regions. In certain embodiments, the highly enriched sequence motif is a nucleotide sequence pattern found within a gene. The highly enriched sequence motif comprises a binding target site for a transcription factor including one or more of the factors REST, ZNF423, HAND1, and NEUROD 1. The highly enriched sequence motifs may comprise one or more of the nucleotide sequences found in SEQ ID NOs: 1-4 (see FIG. 14). Enrichment of these sequence motifs may provide further information regarding the specific subtype or phenotype of the SCLC tumor. In some embodiments, the highly enriched sequence motif is determined using a de novo motif discovery algorithm such as HOMER.

Another aspect of the invention relates to a method of diagnosing SCLC in a subject comprising hybridizing a methylated regions of a genome DNA of a test sample obtained from the subject to a DNA microarray comprising one or a combination of DNA biomarkers, comparing the hybridized methylated regions of the methylated regions from the genome DNA with the hybridization of the corresponding methylated regions of a normal sample or standard sample genome DNA, and predicting that an increase in the methylated regions of the genome DNA hybridizing to the DNA biomarker relative to the methylated regions of the normal sample or standard sample genome DNA hybridizing to the one or a combination of DNA biomarkers indicates that the subject is likely to have SCLC.

Another aspect of the invention relates to a method of treating a subject for SCLC comprises: measuring methylation levels of one or a combination of DNA biomarkers in a test sample from the subject, comparing the methylation levels of the one or the combination of DNA biomarkers with the methylation levels of a corresponding one or a combination of DNA biomarkers in a normal or standard sample, and administering a therapeutically effective amount of chemotherapy to the subject when there is an increase in the methylation levels of the test sample in relation to that of the normal sample or standard sample. In some embodiments, chemotherapy may be administered along with surgery or radiation therapy. In other embodiments, the type of chemotherapy administered may depend on the specific DNA biomarker that is present.

### BRIEF DESCRIPTION OF THE DRAWINGS

FIG. 1 shows a cluster analysis of all samples tested. Cluster analysis was performed for primary small cell lung tumors (T1-18), SCLC cell lines (DMS53, H1688, SW1271, H1447, H1836), normal lung tissue (N1, N2, N3, N5, and N6) and normal human bronchial epithelial cells (HBEC). Spearman correlation was used to derive the dendrograms.

FIG. 2 shows examples of tumor-specific methylation in SCLC. Data are shown for the PROX1, CCDC140, PAX3, and SIM1 genes. The top of the figure indicates the chromosomal coordinates according to the University of California, Santa Cruz (UCSC) Genome browser hg19. Gene names and direction of transcription are shown at the bottom of the figure. The Nimblegen array data (methylated fraction versus input) are shown for three normal lung tissues (N, red) and five primary SCLC tumors (T, green). The methylation signal is shown plotted along the as a P value score. Therefore, the minimum number on the y-axis is 0 (when P=1). The P value score was obtained by NimbleScan software and is derived from the Kolmogorov-Smirnov test comparing the log 2 ratios (MIRA versus input) within a 750 base pair window centered at each probe and the rest of the data on the array.

FIG. 3 shows tumor-specific methylation at the HOXD cluster in SCLC. The top of the figure indicates the chromosomal coordinates according to the UCSC Genome browser hg19. Gene names and direction of transcription are shown at the bottom of the figure. The Nimblegen array data (methylated fraction versus input) are shown for three normal lung tissues (N, red) and five primary SCLC tumors (T, green). The methylation signal is shown plotted along the chromosome as a P value score. The P value score was obtained by NimbleScan software and is derived from the Kolmogorov-Smirnov test comparing the log 2 ratios (MIRA versus input) within a 750 base pair window centered at each probe and the rest of the data on the array.

FIG. 4 illustrates the mapping of tumor-specific methylation peaks in primary SCLC and SCLC cell lines. (a) Localization of the methylation peaks in primary SCLC (6 or more out of 18 tumors methylated; that is, peaks that meet the minimum 80% quantile criterion in 6 of 18 tumors) relative to gene position; (b) Localization of the methylation peaks in primary SCLC (14 or more out of 18 tumors methylated) relative to gene position; (c) Localization of the methylation peaks in SCLC cell lines (4 or more out of 5 cell lines methylated) relative to gene position; (d) Overlap of methylation peaks between SCLC primary tumors (6 or more out of 18 tumors methylated) and SCLC cell lines (4 or more out of 5 cell lines methylated); (e) Overlap of methylation peaks between SCLC primary tumors (14 or more out of 18 tumors methylated) and SCLC cell lines (4 or more out of 5 cell lines methylated); and (f) Cluster analysis of methylation peaks. Methylation peaks found in at least 33% of tumor samples but not in normal samples were identified. Then the data were subjected to hierarchical clustering with Euclidean distance and average linkage method using Cluster v3.0 (de Hoon et al., 2004) and visualized in Java TreeView (Saldanha, A., 2004). Red, methylated state; green, unmethylated state.

FIG. 5 provides a list of the gene targets methylated in 77% or more of primary SCLCs. The chromosome number, starting peak, ending peak, hgnc symbol, and description of each gene are provided. The ^{a} symbol next to the RASSF1A gene indicates a previously validated gene with a lower threshold for normal tissues than that used for the other regions.

FIG. 6 shows the validation of gene-specific methylation in SCLC by COBRA assays. COBRA analysis of the DMRTA2, GALNTL1 and MIR129-2 genes in normal lung (N) and primary SCLC (T). Matched tumor-normal sample pairs were N1 and T16, N2 and T17, and N3 and T18.

FIG. 7 shows the validation of gene-specific methylation in SCLC by bisulfite sequencing analysis of the DMRTA2 and GALNTL1 genes. Two normal lung and four SCLCs were analyzed. Open circles indicate unmethylated CpG sites; closed circles show methylated CpG sites.

FIG. 8 illustrates the functional annotation and motif finding analysis. (a) DAVID functional analysis clusters that contained the highest enrichment scores in all three categories: 33% or more of tumors, 77% or more of tumors and cell lines; and (b) Motif finding analysis. Significantly enriched consensus motifs for REST (SEQ ID NO: 1), ZNF423 (SEQ ID NO: 2), Hand1 (SEQ ID NO: 3) and NEUROD1 (SEQ ID NO: 4) are shown.

FIG. 9 shows a table of homeobox genes methylated at the promoter in 33% or more of primary SCLCs.

FIG. 10 shows a table of homeobox genes methylated within the gene body of 33% or more of primary SCLCs.

FIG. 11 shows examples of tumor-specific methylation of NEUROD1 target genes in SCLC. The top of the figure indicates the chromosomal coordinates according to the UCSC Genome browser hg19. Gene names and direction of transcription are shown at the bottom of the figure. The Nimblegen array data (methylated fraction versus input) are shown for three normal lung tissues (red) and five primary SCLC tumors (blue). The methylation signal is shown plotted along the chromosome as a P-value score. Therefore, the minimum number on the y axis is 0 (when P=1). The P-value score was obtained by the NimbleScan software and is derived from the Kolmogorov—Smirnov test comparing the log 2 ratios (MIRA versus input) within a 750-bp window centered at each probe and the rest of the data on the array. The asterisks indicate the location of the NEUROD1 target sites.

FIG. 12 shows the mRNA expression of NEUROD1 in SCLC cell lines. SCLC and HBEC cell lines were cultured to ˜90% confluence in 35-mm dishes. Total RNA was isolated, and mRNA expression of NEUROD1 was determined by real-time RT-PCR, normalized to 18s RNA and expressed relative to HBEC levels. One of the SCLC cell lines (DMS53) was transiently transfected with a NEUROD1 expression plasmid to validate the NEUROD1 RT-PCR method. Expression of NEUROD1 was extremely low in all samples, except for the one with NEUROD1 overexpression, as indicated by ct values of ≧40. Values are the averages of three independent experiments.

FIG. 13 shows the methylation at the promoters of NEUROD1, HAND1, REST, and ZNF423 in SCLC. Methylation data are shown for normal lung and primary tumors (A) and HBEC and SCLC cell lines (B). The position of the transcription start sites and the direction of transcription are indicated by arrows.

FIG. 14 shows the DNA nucleotide sequences for SEQ ID NOs: 1, 2, 3, and 4. SEQ ID NO: 1 is the highly enriched sequence motif for REST; SEQ ID NO: 2 is the highly enriched sequence motif for ZN423; SEQ ID NO: 3 is the highly enriched sequence motif for Hand1; and SEQ ID NO: 4 is the highly enriched sequence motif for NEUROD1. A represents the DNA nucleotide adenine, G represents the nucleotide guanine, C represents the nucleotide cytosine, and T represents the nucleotide thymine. X¹ may represent either a C or G, X² represents either a T or A, X³ represents either an A or C, and X⁴ represents either an A or T.

### DETAILED DESCRIPTION

One aspect of the invention relates to a method for the identification of novel DNA biomarkers and the use of the aberrant methylation patterns of the DNA biomarkers to diagnose small cell lung cancer (SCLC) in a subject. SCLC is a disease characterized by aggressive clinical behavior and lack of effective therapy. Due to its tendency for early dissemination, only a third of patients have limited-stage disease at the time of diagnosis. SCLC is thought to derive from pulmonary neuroendocrine cells. As described in the Examples below, methylation profiling with the methylated CpG island recovery assay (MIRA) (Rauch and Pfeifer, 2009; Rauch and Wu et al., 2009) was used in combination with microarrays to conduct the first genome-scale analysis of methylation changes that occur in primary SCLC and SCLC cell lines. Among the hundreds of tumor-specifically methylated genes discovered, 73 gene targets that were methylated were identified in more than 77% of primary SCLC tumors, most of which have never been linked to aberrant methylation in tumors. These methylated targets in such a large fraction of the patient population may be of particular value for designing DNA methylation-based biomarkers for early detection of SCLC, for example, in serum or sputum, and for disease management. Additionally, motif analysis of tumor-specific methylated regions identified methylation at binding sites for several neural cell fate-specifying transcription factors. DNA methylation at these sites may prevent transcription factor binding, leading to the inhibition of active transcription or the recruitment of methyl-binding proteins, causing gene suppression and guiding the cell toward a malignant state. As a result, these highly enriched sequence motifs may help to differentiate between different subtypes and phenotypes of SCLC, which may be useful during therapeutic management of SCLC.

Methods of Diagnosing or Predicting SCLC

One aspect of the invention relates to a method of diagnosing, determining or predicting that a subject has small cell lung cancer (SCLC) that is associated with aberrant methylation of DNA in a sample are provided, wherein the method by measuring methylation levels of one or a combination of DNA biomarkers in a test sample from a subject, and comparing the methylation levels of the one or the combination of DNA biomarkers with the methylation levels of a corresponding one or a combination of DNA biomarkers in a normal sample or standard sample, and predicting that an increase in the methylation levels of the test sample in relation to that of the normal sample or standard sample indicates that the subject is likely to have SCLC. The aberrant methylation as referred to herein is hypermethylation. In some embodiments, the method further includes obtaining the test sample from the subject. According to some embodiments, a test sample is an organ, a fragment of organ, a tissue, a fragment of a tissue, body fluid, blood, serum, urine, sputum, which may or may not have a condition or a disease. In some embodiments, the test sample is a specimen obtained by bronchoscopy or a bronchioalveolar sample.

As used herein, a “subject” refers to a human or animal, including all mammals such as primates (particularly higher primates), sheep, dog, rodents (e.g., mouse or rat), guinea pig, goat, pig, cat, rabbit, and cow. In some embodiments, the subject is a human. In other embodiments, the subject is a human that may be considered at high-risk for developing SCLC, including an individual who may be a current or former smoker. In certain embodiments, the subject is suffering from SCLC.

Another aspect of the invention relates to a method of diagnosing SCLC in a subject comprising hybridizing a methylated regions of a genome DNA of a test sample obtained from the subject to a DNA microarray comprising one or a combination of DNA biomarkers, comparing the hybridized methylated regions of the methylated regions from the genome DNA with the hybridization of the corresponding methylated regions of a normal sample or standard sample genome DNA, and predicting that an increase in the methylated regions of the genome DNA hybridizing to the DNA biomarker relative to the methylated regions of the normal sample or standard sample genome DNA hybridizing to the one or a combination of DNA biomarkers indicates that the subject is likely to have SCLC.

In certain embodiments, the method further includes one or more of the following steps: obtaining the test sample from the subject as described supra, obtaining the genome DNA from the test sample from the subject, and obtaining methylated regions from the genome DNA.

As used herein, the DNA biomarkers are fragments of a polynucleotide (e.g., regions of genome polynucleotide or DNA) which likely contain CpG island(s), or fragments which are more susceptible to methylation or demethylation than other regions of genome DNA. The term “CpG islands” is a region of genome DNA which shows higher frequency of 5′-CG-3′ (CpG) dinucleotides than other regions of genome DNA. Methylation of DNA at CpG dinucleotides, in particular, the addition of a methyl group to position 5 of the cytosine ring at CpG dinucleotides, is one of the epigenetic modifications in mammalian cells. CpG islands often harbor the promoters of genes and play a pivotal role in the control of gene expression. CpG islands are usually unmethylated in normal tissues, but a subset of islands becomes methylated during the development of a disease (e.g., tumor development). It has been reported that changes in DNA methylation patterns occur in a developmental stage and tissue specific manner and often accompany tumor development, most notably in the form of CpG island hypermethylation. During tumorigenesis, both alleles of a tumor suppressor gene need to be inactivated by genomic changes such as chromosomal deletions or loss-of-function mutations in the coding region of a gene. As an alternative mechanism, transcriptional silencing by hypermethylation of CpG islands spanning the promoter regions of tumor suppressor genes is a common and important process in carcinogenesis. Since hypermethylation generally leads to inactivation of gene expression, this epigenetic alteration is considered to be a key mechanism for long-term silencing of tumor suppressor genes. The importance of promoter methylation in functional inactivation of lung cancer suppressor genes is becoming increasingly recognized. It is estimated that between 0.5% and 3% of all genes carrying CpG islands may be silenced by DNA methylation in lung cancer (Costello et al., 2000). The aberrant methylation referred to herein is hypermethylation. DNA biomarkers as described herein may include one or a combination of DNA biomarkers.

It is contemplated that the DNA biomarkers that are hypermethylated, as referred to herein, may be unmethylated in a normal sample or standard sample (e.g., normal or control tissue without disease, or normal or control body fluid, blood, serum, urine, sputum), most importantly, in the healthy tissue the tumor originates from and/or in healthy blood, serum, urine, sputum or other body fluid. In some embodiments, a normal sample or standard sample may comprise normal bronchial epithelial cells (HBECs). In other embodiments, the normal sample or control sample may be from a different subject other than from whom the test sample was obtained. In certain embodiments, the test sample is subject to diagnosing methods to determine the methylation levels of at least one DNA biomarker from the test sample in comparison to that of a normal or standard sample.

In some embodiments, the DNA biomarker in the test sample is heavily methylated in a large fraction of the tumors at a methylation frequency of ≧ about 50% or ≧ about 60%, ≧ about 70%, ≧ about 75%, ≧ about 80%, ≧ about 85%, ≧ about 90%, ≧ about 95%, or about 100%. In other embodiments, the DNA biomarker is methylated at a frequency of greater than about 77% of SCLC tumors. In certain embodiments, the DNA biomarker is methylated at a frequency of greater than about 33% of SCLC tumors. In one embodiment, when methylation of the DNA biomarkers is analyzed, a cutoff of 80% is used to determine hypermethylated or methylated regions and a cutoff below 56% is used to determine unmethylated regions. In some embodiments, a stringent tumor-specific methylated region may be defined as the overlapping region that meets the minimum 80% quantile criterion in at least about ≧77% of tumors and is below the quantile in at least about 56% of normal tissues. In certain embodiments, a less stringent set may be defined as an overlap between at least about 33% of SCLC tumors. In certain embodiments, the signal in the tumor is at least 2 or 3-fold greater than the signal in the normal tissues.

According to certain embodiments, DNA biomarkers that are methylated include those genes disclosed in FIG. 5. In certain embodiments, select DNA biomarkers from FIG. 5 include the genes HMX2, ONECUT1, FOXE1, TBX4, GSC, VGLL2, NKX2-1, WT1-AS, SOX1, ZBTB43, GHSR, EOMES, HCK, KLHL6, TBX5, CDH22, IFNA12P, TAC1, CCDC140, HIST1H4I, ZNF560, EVX1, NKX3-2, TRPC5, NKX2-5, RESP18, LMX1A, EN1, AVPR1A, GDF6, TAL1, SLITRK1, GDNF, PAX5, C14orf23, NXPH1, OPRM1, CDH26, C9orf53, CBLN1, MIR124-1, NKX2-2, TLX3, HOXD4, NKX6-1, MIR1469, MIR9-3, and RASSF1A. The methylation of these biomarkers may occur at a frequency of greater than about 77% of SCLC tumors.

As described further in the Examples below, eleven methylated genomic regions, which were predicted by the array analysis, were randomly selected and validated using bisulfite-based COBRA assays. The validated targets fell into various major functional categories, including transcription factors and noncoding RNAs. Validation of this set of samples revealed the specificity of the array analysis. In some embodiments, DNA biomarkers that are methylated include the genes GALNTL1, MIR-10A, MIR-129-2, MIR-196A2, MIR-615, MIR-9-3, AMBRA1, HOXD10, PROX1, ZNF672 and DMRTA2. In certain embodiments, the DNA biomarker includes the gene DMRTA2 which may be methylated at a frequency of at least about 94% of SCLC tumors.

In another embodiment, DNA biomarkers include genes involved in neuronal or neuroendocrine differentiation found in at least about 77% of SCLC tumors. In some embodiments, DNA biomarkers may include one or more genes listed in Table 6. Examples of these DNA biomarkers are the genes EOMES/TBR2, TAC1, and RESP18.

Additionally, DNA biomarkers that can differentiate between different subtypes or tumor entities, or are of prognostic significance, would be of great value. Specific DNA methylation patterns may distinguish tumors with low and high metastatic potential making it possible to apply optimal treatment regimens early. Aberrant methylation at important regulatory regions such as transcription factor binding sites may also be an indicator of SCLC and may help differentiate between different subtypes and phenotypes of SCLC. A potential way of disrupting cell fate decisions is not by merely reducing the responsible transcription factors but by altering the selectivity toward their genomic recognition sites by aberrant methylation at these regulatory regions, leading to the prevention of binding. It has been known that DNA methylation can prevent transcription factor binding leading to the inhibition of active transcription or the recruitment of methyl-binding proteins, causing gene suppression (Suzuki et al., 2008). When looking for binding sites of important cell fate specificators in the SCLC tumor-specific methylated regions as demonstrated in the Examples described below, such a correlation was identified, especially concerning the transcription factors NEUROD1, ZNF423, HAND1, and REST (FIG. 8b).

Thus, in some embodiments, a DNA biomarker may comprise a sequence motif that is identified in regions that are specifically methylated in SCLC tumor samples. The sequence motif may be highly enriched compared with non-tumor specifically methylated regions. The enrichment may be characterized by P-values (e.g. as listed in the second column of FIG. 8b) the smaller the P-value, the higher is the enrichment. In certain embodiments, the highly enriched sequence motif is a nucleotide sequence pattern found within a methylated gene region comprising a binding target site for a transcription factor. In certain embodiments, the transcription factor may be REST, ZNF423, HAND1, and/or NEUROD1. In other embodiments, the gene that is methylated is a DNA biomarker that is involved in cell fate commitment and comprises NEUROD1- or HAND 1-binding sites. Examples of these genes are GDNF, NKX2-2, NKX6-1, EVX1, and SIM2. In one embodiment, the gene region that is methylated is the promoter region of the transcription factor. In some embodiments, the DNA biomarker is methylated at a frequency of at least about 33% of SCLC tumors.

In one embodiment, if the transcription factor is REST, then the highly enriched sequence motif within the DNA biomarker may comprise the DNA nucleotide consensus sequence: X¹⁻T-G-X²-X³-C-A-X⁴⁻G-G-T-G-C-T-G-A (SEQ ID NO: 1), where X¹ can be either C or G, X² can be either T or A, X³ can be either A or C, and X⁴ can be either A or T (see FIGS. 8b and 14). In another embodiment, if the transcription factor is ZNF423, then the highly enriched sequence motif within the DNA biomarker may comprise the nucleotide consensus sequence: G-A-A-C-C-C-T-G-C-G-G-G-T-C (SEQ ID NO: 2) (FIGS. 8b and 14). In some embodiments, if the transcription factor is HAND1, then the highly enriched sequence motif within the DNA biomarker may comprise the nucleotide consensus sequence: C-C-A-G-A-C-C-G-C-A-G-A-A-A (SEQ ID NO: 3) (FIGS. 8b and 14). In still another embodiment, if the transcription factor is NEUROD1, then the DNA biomarker may be a gene such as PITX2, GDNF, and NKX2-2 (FIG. 11). Additionally, if the transcription factor is NEUROD1, then the highly enriched sequence motif within the DNA biomarker may comprise the nucleotide consensus sequence: C-A-G-A-T-T-G-C-T-A (SEQ ID NO: 4) (FIGS. 8b and 14). The highly enriched sequence motif may be identified using a de novo motif discovery algorithm. In one embodiment, the algorithm that is used to identify the highly enriched sequence motif is HOMER. In some embodiments, highly enriched sequence motifs of 8-30 base pairs in length may be identified with the highest alignments to known transcription factors. In other embodiments, a highly enriched sequence motif may have up to two nucleotide mismatches with any of the sequences provided by SEQ ID NOs: 1-4 (FIG. 14).

There are a number of methods that can be employed to determine, identify, and characterize methylation or aberrant methylation of a region/fragment of DNA or a region/fragment of genome DNA (e.g., CpG island-containing region/fragment) in the development of SCLC (e.g., tumorigenesis) and thus diagnose the onset, presence or status of SCLC.

In another embodiment, a methylation detection technique is based on restriction endonuclease cleavage. These techniques require the presence of methylated cytosine residues within the recognition sequence that affect the cleavage activity of restriction endonucleases (e.g., HpaII, HhaI) (Singer et al., 1979). Southern blot hybridization and polymerase chain reaction (PCR)-based techniques may also be used with along with this approach.

In another embodiment, a methylation detection technique is based on the differential sensitivity of cytosine and 5-methylcytosine towards chemical modification (e.g., bisulfite dependent modification) and/or cleavage. This methodology allows single base resolution. In one example, hydrazine modification, as developed for Maxam-Gilbert chemical DNA sequencing, may be used to distinguish cytosines from methylcytosines with which it does not react (Pfeifer et al., 1989). The principle of bisulfite genomic sequencing is that methylated and unmethylated cytosine residues react in a different manner with sodium bisulfite (Clark et al., 1994). After bisulfite treatment of genomic DNA, the unmethylated cytosines are converted to uracils by hydrolytic deamination, while methylated cytosine residues remain unchanged and do not react with sodium bisulfite and therefore remain intact. After this chemical treatment resulting in cytosine deamination, the region of interest must be PCR amplified with primers complementary to the deaminated uracil-containing sequence, and in most cases the PCR products are cloned and then sequenced.

In another embodiment, the combined bisulfite-restriction analysis (COBRA assay) is a bisulfite dependent methylation assay, which may be used to detect methylation. PCR products obtained from bisulfite-treated DNA can also be analyzed by using restriction enzymes that recognize sequences containing 5′CG, such as TaqI (5′TCGA) or BstUI (5′CGCG) such that methylated and unmethylated DNA can be distinguished (Xiong and Laird, 1997).

In one embodiment, another bisulfite dependent methylation assay that may be used to detect methylation is the methylation-specific PCR assay (MSP) (Herman et al., 1996). Sodium bisulfite treated genomic DNA serves as the template for a subsequent PCR reaction. Specific sets of PCR primers are designed in such a way to discriminate between bisulfite modified and unmodified template DNA and between unmethylated (deaminated) and methylated (non-deaminated) cytosines at CpG sites.

In some embodiments, a methylation detection technique that may be used is based on the ability of the MBD domain of the MeCP2 protein to selectively bind to methylated DNA sequences (Fraga et al., 2003). The bacterially expressed and purified His-tagged methyl-CpG-binding domain is immobilized to a solid matrix and used for preparative column chromatography to isolate highly methylated DNA sequences. Genomic DNA is loaded onto the affinity column and methylated-CpG island-enriched fractions are eluted by a linear gradient of sodium chloride. PCR or Southern hybridization techniques may be used to detect specific sequences in these fractions.

In another embodiment, a methylation detection technique that may be used is known as methyl-CpG island recovery assay (MIRA), which is based on the fact that the MBD2b protein can specifically recognize methylated-CpG dinucleotides. This interaction is enhanced by the MBD3L1 protein. Matrix-assisted binding and simple PCR assays are used to detect methylated DNA sequences in the recovered fraction. MIRA does not depend on the use of sodium bisulfite but has similar sensitivity and specificity as bisulfite-based approaches (Rauch and Pfeifer, 2005). Briefly, Methyl-CpG binding domain (MBD) proteins, such as MBD2, have the capacity to bind specifically to methylated DNA sequences. Among the MBD proteins, MBD2b, the short protein isoform translated from the MBD2 mRNA, has been shown to have strong affinity for methylated DNA and the highest capacity to discriminate between methylated and unmethylated DNA, in a relatively sequence-independent manner. MBD2b forms a heterodimer with a related protein, MBD3L1, which further increases the affinity of MBD2b for methylated DNA. In the MIRA procedure, sonicated or restriction-cut genomic DNA, isolated from different cells or tissues, is incubated with the complex of GST-MBD2b and His-MBD3L1 bound to glutathione-agarose. These two recombinant proteins can easily be expressed in E. coli. Specifically bound DNA is eluted from the matrix and gene-specific PCR reactions can be performed to detect CpG island methylation. Methylation can be detected using 1 ng of DNA or 3,000 cells. MIRA has a high specificity for enriching the methylated DNA and unmethylated DNA molecules that stay in the supernatant.

The MIRA assay has a high specificity to detect the methylated CpG island-containing fraction/region/fragment of the genome DNA. In one embodiment, MIRA-assisted microarray analysis may be employed to determine DNA methylation patterns or diagnose SCLC associated with aberrant methylation of DNA biomarkers or CpG containing regions/fragments (Rauch et al., 2006). The MIRA procedure has been applied to isolate the methylated CpG island fraction from SCLC tumor cell lines and SCLC primary tumors. In some embodiments, enrichment of the methylated double-stranded DNA fraction by MIRA may be performed as described previously in Rauch and Pfeifer, 2009 and Rauch and Wu et al., 2009, which is hereby incorporated by reference. Various types of microarrays can be used in analyzing DNA methylation patterns on a genome-wide scale. MIRA is compatible with Affymetrix promoter arrays as well as with Agilent and NimbleGen arrays. In further embodiments, the labeling of amplicons, microarray hybridization and scanning may be performed according to the NimbleGen (Madison, Wis., USA) protocol, which is hereby incorporated by reference. Additionally, NimbleGen tiling arrays may be used for hybridization and the MIRA-enriched DNA may be compared with the input DNA.

According to some embodiments, the MIRA technique may be used in combination with microarray analysis. Analysis of the arrays may be performed with R version 2.10, Perl scripts and the Bioconductor package Ringo. A quantile-based approach may be chosen to estimate methylation intensities instead of estimating a cutoff ratio based on a hypothetical normal distribution for non-bound probes (Ringo). In some embodiments, a quantile range of at least about 80% may be chosen as a cutoff for methylated DNA biomarkers (these may be defined as hypermethylated regions). In one embodiment, a COBRA analysis may be used to validate predicted peaks in different samples. Certain embodiments may define tumor specific methylated regions of DNA biomarkers by analyzing the data using different levels of stringencies including least stringent and more stringent analyses. In one embodiment, for the least stringent analysis, an overlap of peaks in at least about 33% of samples may be required above the cutoff quantile threshold of at least about 80%. For example, as described further in Example 1 below, 6 or more out of 18 tumor samples (33%) was required above the cutoff quantile threshold of 80%; the genomic regions were defined and for those regions only one out of five normal tissues was allowed to overlap with a peak called on a 56% basis, which resulted in an at least 1.5 ratio change. Overlaps may be calculated using BEDtools (Quinlan et al., 2010). Alternatively, in another embodiment, a more stringent analysis may be used wherein an overlap of peaks in at least 77% of samples may be required above the cutoff quantile threshold of 80%. As demonstrated in Example 1 below, an overlap of peaks in at least 14 out of 18 tumors (>77%) was required with the same settings as the least stringent analysis.

Methods have been developed to analyze DNA methylation patterns on a genome-wide scale that can be used in the embodiments described herein. These methods include, for example, 1) restriction landmark genomic scanning, 2) methylation-sensitive representational difference analysis, 3) arbitrarily-primed PCR, 4) differential methylation hybridization in combination with a CpG island microarray (methods 1-4 use methylation sensitive restriction, 5) expression microarrays to look for genes reactivated by treatment with DNA methylation inhibitors, e.g. 5-aza-deoxycytidine, 6) genomic tiling and BAC microarrays, 7) immunoprecipitation using antibody against 5-methylcytosine combined with microarrays, 8) chromatin immunoprecipitation with antibodies against methyl-CpG binding proteins, 9) the use of the methylation-dependent restriction enzyme McrBC to cleave methylated DNA, and 10) direct sequencing of bisulfite-converted genomes (See Pfeifer at el., 2007, for review).

Another aspect of the invention relates to a method of treating a subject suffering from SCLC by administering chemotherapy based on the presence of novel, methylated DNA biomarkers.

Methods of Treating SCLC

According to some embodiments, the methods described herein may be used to treat, optimally treat, or therapeutically manage subjects with SCLC. The method comprises measuring methylation levels of one or a combination of DNA biomarkers in a test sample of the subject, comparing the methylation levels of the one or a combination of DNA biomarkers with the methylation levels of a corresponding one or combination of DNA biomarkers in a normal sample or standard sample, and administering a therapeutically effective amount of chemotherapy to the subject when there is an increase in the methylation levels of the test sample in relation to that of the normal sample or standard sample. The test sample and normal sample or standard sample are the samples as described above. In certain embodiments, the subject is suffering from SCLC.

According to certain embodiments, the DNA methylation biomarkers may be any one or a combination of the DNA biomarkers as described above. For example, in some embodiments, the DNA biomarker may comprise a highly enriched sequence motif comprising a transcription factor binding site. In one embodiment, the methods described herein may be used to screen subjects considered to be at high-risk for SCLC, including individuals that are current or former smokers. In another embodiment, the presence or absence of a methylation biomarker (as listed in FIG. 5) or the presence of a highly enriched sequence motif that is identified may be used to distinguish the particular subtype or phenotype of the SCLC tumor. In another embodiment, the methods may include steps used to therapeutically manage the treatment of subjects suffering from SCLC based on the presence of particular methylated DNA biomarkers. In another embodiment, a method of optimally treating a subject for SCLC includes administering a particular chemotherapy based on the highly enriched sequence motif.

The chemotherapy used in accordance with the methods described herein may be administered, by any suitable route of administration, alone or as part of a pharmaceutical composition. A route of administration may refer to any administration pathway known in the art, including but not limited to aerosol, enteral, nasal, ophthalmic, oral, parenteral, rectal, transdermal (e.g., topical cream or ointment, patch), or vaginal. “Transdermal” administration may be accomplished using a topical cream or ointment or by means of a transdermal patch.

“Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal.

The term “effective amount” as used herein refers to an amount of a chemotherapy that produces a desired effect. For example, a population of cells may be contacted with an effective amount of chemotherapy to study its effect in vitro (e.g., cell culture) or to produce a desired therapeutic effect ex vivo or in vitro. An effective amount of chemotherapy may be used to produce a therapeutic effect in a subject, such as preventing or treating a target condition, alleviating symptoms associated with the condition, or producing a desired physiological effect. In such a case, the effective amount of a chemotherapy is a “therapeutically effective amount,” “therapeutically effective concentration” or “therapeutically effective dose.” The precise effective amount or therapeutically effective amount is an amount of the chemotherapy that will yield the most effective results in terms of efficacy of treatment in a given subject or population of cells. This amount will vary depending upon a variety of factors, including but not limited to the characteristics of the chemotherapy (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the chemotherapy is administered alone or in combination with another chemotherapy, drug, therapy or other therapeutic method or modality. One skilled in the clinical and pharmacological arts will be able to determine an effective amount or therapeutically effective amount through routine experimentation, namely by monitoring a cell's or subject's response to administration of a chemotherapy and adjusting the dosage accordingly. For additional guidance, see Remington: The Science and Practice of Pharmacy, 21^{st} Edition, Univ. of Sciences in Philadelphia (USIP), Lippincott Williams & Wilkins, Philadelphia, Pa., 2005, which is hereby incorporated by reference as if fully set forth herein.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

In certain embodiments, the therapeutically effective amount of chemotherapy administered to the subject when there is an increase in the methylation levels of the test sample in relation to that of the normal sample or standard sample is such that the administration slows down, alleviate, or prevents the methylation levels increase of future test samples of the subject determined according to the methods disclosed herein.

In some embodiments, the chemotherapy that is administered may include any chemotherapy that is used to treat SCLC such as Abitrexate (Methotrexate), Etopophos (Etoposide Phosphate), Folex (Methotrexate), Folex PFS (Methotrexate), Hycamtin (Topotecan Hydrochloride), Methotrexate, Methotrexate LFP, Mexate (Methotrexate), Mexate-AQ (Methotrexate), Toposar (Etoposide), Topotecan (Hydrochloride), and VePesid (Etoposide). Additionally, in some embodiments, the chemotherapy that is administered may be used in conjunction with surgery or radiation therapy.

In some embodiments, the chemotherapy described above may be administered in combination with one or more additional therapeutic agents. “In combination” or “in combination with,” as used herein, means in the course of treating the same disease in the same patient using two or more agents, drugs, treatment regimens, treatment modalities or a combination thereof, in any order. This includes simultaneous administration, as well as in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

Examples of therapeutic agents that may be administered in combination with the chemotherapy include, but are not limited to, other chemotherapeutic agents, therapeutic antibodies and fragments thereof, toxins, radioisotopes, enzymes (e.g., enzymes to cleave prodrugs to a cytotoxic agent at the site of the tumor), nucleases, hormones, immunomodulators, antisense oligonucleotides, nucleic acid molecules (e.g., mRNA molecules, cDNA molecules or RNAi molecules such as siRNA or shRNA), chelators, boron compounds, photoactive agents and dyes. The therapeutic agent may also include a metal, metal alloy, intermetallic or core-shell nanoparticle bound to a chelator that acts as a radiosensitizer to render the targeted cells more sensitive to radiation therapy as compared to healthy cells.

Chemotherapeutic agents that may be used in accordance with the embodiments described herein are often cytotoxic or cytostatic in nature and may include, but are not limited to, alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as therapeutic agents in accordance with the embodiments of the disclosure include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte—colony stimulating factor, granulocyte macrophage-colony stimulating factor, hexamethylmelamine, hydrocortisone hydroxyurea, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

Therapeutic antibodies and functional fragments thereof, that may be used as therapeutic agents in accordance with the embodiments of the disclosure include, but are not limited to, alemtuzumab, bevacizumab, cetuximab, edrecolomab, gemtuzumab, ibritumomab tiuxetan, panitumumab, rituximab, tositumomab, and trastuzumab and other antibodies associated with specific diseases listed herein.

Toxins that may be used as therapeutic agents in accordance with the embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Radioisotopes that may be used as therapeutic agents in accordance with the embodiments of the disclosure include, but are not limited to, ³²P, ⁸⁹Sr, ⁹⁰Y, ^{99m}Tc, ⁹⁹Mo, ¹³¹I, ¹⁵³Sm, ¹⁷⁷Lu, ¹⁸⁶Re, ²¹³Bi, 5 ²²³Ra and ²²⁵Ac.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entireties, as if fully set forth herein.

### EXAMPLES

### Example 1

### DNA Methylation Analyses of Small Cell Lung Cancer Primary Tumors and Cell Lines

The MIRA technique, used in combination with microarray analysis, was a high-resolution mapping technique and had proven successful for profiling global DNA methylation patterns in non-small cell lung cancer (NSCLC) and other tumors (Rauch et al., 2008; Wu et al., 2010; Rauch et al., 2007; Tommasi et al., 2009). As described in this and other Examples below, this sensitive method was used to study the methylation status of CpG islands and promoters in small cell lung cancer (SCLC) to investigate the potential role of methylation changes in the initiation and development of SCLC, as well as to discover potential biomarkers for better management of the disease.

Identification of Methylated Genes in Human SCLC Tissue on a Genome-Wide Platform.

Eighteen human primary SCLC and five SCLC cell line DNA samples were screened for methylation by MIRA-based microarrays. DNAs from five normal healthy lung tissues adjacent to the tumor and obtained at the time of surgical resection were used as controls in the MIRA analysis. DNA was subjected to MIRA enrichment as described previously (Rauch and Pfeifer, 2009; Rauch and Wu et al., 2009) and subsequent microarray analysis was performed on 720k Nimblegen CpG island plus promoter arrays.

Microarray Data Analysis.

To increase the specificity of MIRA-based enrichment signals, peaks were identified based on different quantiles of four neighboring probes. Peaks were then calculated using the base functions of the Bioconductor package Ringo (Toedling et al., 2007). Table 1 shows the specificity and sensitivity of this approach relative to different quantile ranges using DNA from the SCLC cell line SW1271.

TABLE 1
Validation of microarray results by COBRA assays.

| Top | No. of | | | | | |
|---|---|---|---|---|---|---|
| Quantile | targets | | | | | |
| (%) | tested^{a} | Met | UnMet | PCR fails | % Met | % UnMet |
| 99 | 10 | 9 | — | 1 | 100 | 0 |
| 95 | 10 | 9 | — | 1 | 100 | 0 |
| 90 | 10 | 9 | 1 | — | 90 | 10 |
| 85 | 10 | 9 | 1 | — | 90 | 10 |
| 80 | 10 | 7 | 3 | — | 70 | 30 |
| 70 | 14 | 3 | 5 | 6 | 37.5 | 62.5 |
| 60 | 19 | 3 | 12 | 4 | 20 | 80 |
| 50 | 13 | 2 | 11 | — | 15 | 85 |

Abbreviations:
COBRA, combined bisulfite restriction analysis;
Met, methylated;
UnMet, unmethylated.
^{a}COBRA was performed for each quantile category with bisulfite-converted DNA from the SW1271 cell line. Results were tabulated for number of Met and UnMet genes in these various categories.

Based on the validations conducted by combined bisulfite restriction analysis (COBRA) single-gene methylation assays, a cutoff of 80% was chosen for medium to strongly methylated regions and a cutoff below 56% was defined as not methylated. Thus, compared with the conventional NimbleScan method using the default settings, the sensitivity of methylation peak detection could be increased to 94% without decreasing specificity. As this threshold was defined for one SCLC cell line, the same settings were tested for primary small lung cancer samples and a significant increase of false positive predicted hypermethylated regions was not observed.

Using the peak identification algorithm described below in the Materials and Methods section, ˜15 000 methylation peaks were identified in each sample (Table 2).

TABLE 2
Total number of methylation peaks
identified in individual samples.

| | Number of |
|---|---|
| Sample | methylation peaks |
| T1 | 15185 |
| T2 | 15225 |
| T3 | 15404 |
| T4 | 14804 |
| T5 | 15254 |
| T6 | 15414 |
| T7 | 14906 |
| T8 | 15236 |
| T9 | 15353 |
| T10 | 15902 |
| T11 | 15338 |
| T12 | 15629 |
| T13 | 15635 |
| T14 | 15204 |
| T15 | 14843 |
| T16 | 15340 |
| T17 | 14539 |
| T18 | 15584 |
| N1 | 15366 |
| N2 | 15336 |
| N3 | 15299 |
| N5 | 14584 |
| N6 | 15413 |
| HBEC | 15542 |
| DMS53 | 15379 |
| H1417 | 15423 |
| H1688 | 15214 |
| H1836 | 15405 |
| SW1271 | 14952 |

A clustering analysis of tumor samples and controls showed that SCLC cell lines clustered together and that four of the five normal samples were close to each other, but different tumor samples occupied different spaces in the dendrogram (FIG. 1).

Taking into account that 18 tumor samples and 5 normal samples were used for microarray data analysis, a stringent tumor-specific methylated region was defined as the overlapping region that met the minimum 80% quantile criterion in 14 of 18 tumors and was below the 56% quantile in 4 of 5 normal tissues. A less stringent set was defined as an overlap between at least 6 peaks from tumor samples out of 18, using the same criteria as above. Thus, the comparison was focused mainly on strongly methylated regions versus poorly methylated regions. Although small methylation level differences could not be detected this way, the aim of discovering uniquely strongly methylated and tumor specific regions was well supported by this approach.

Methylated Genes in Primary SCLC.

FIG. 2 shows examples of tumor-specific methylation peaks at the PROX1, CCDC140, PAX3 and SIM1 genes located on chromosomes 1, 2 and 6, respectively. FIG. 3 shows extensive tumor-specific methylation of the HOXD cluster on chromosome 2. Compilation of tumor-specific methylation peaks revealed a total of 698 regions in 6 out of 18 tumors (≧33% of SCLC tumors) compared with normal lung DNA, which represented 339 ensemb1 gene IDs for promoter-related tumor-specifically methylated regions (defined as −5000 to +1000 relative to the TSS), 197 ensemb1 gene IDs related to peaks mapped to the gene bodies and 63 ensemb1 gene IDs for peaks mapped downstream of the corresponding genes (FIG. 4a). Individual primary SCLCs contained between 366 and almost 1500 tumor specific methylation peaks (Table 3).

TABLE 3
Total number of tumor-specific methylation peaks
in individual SCLC tumors and cell lines.

| Sample | Number of tumor-specific peaks |
|---|---|
| T1 | 1207 |
| T2 | 1312 |
| T3 | 1085 |
| T4 | 1346 |
| T5 | 1277 |
| T6 | 1113 |
| T7 | 1351 |
| T8 | 1261 |
| T9 | 1118 |
| T10 | 1386 |
| T11 | 1141 |
| T12 | 1016 |
| T13 | 1258 |
| T14 | 1489 |
| T15 | 366 |
| T17 | 1122 |
| T18 | 518 |
| DMS54 | 3577 |
| H1417 | 3189 |
| H1688 | 4485 |
| H1836 | 3212 |
| SW1271 | 2779 |

There were 73 tumor-specific methylated peaks, which were found in at least 14 out of 18 SCLC tumors (>77% of SCLC tumors), that corresponded to 28 ensemb1 gene IDs for promoters, 30 ensemb1 gene IDs for gene bodies and 11 for downstream regions (FIG. 4b). These methylated genes from 77% or more of the SCLC tumors are presented in FIG. 5.

Identification of Methylated Genes in Human SCLC Lines.

Owing to the limited availability of primary SCLC tissue, several SCLC cell lines originally derived from primary tumor sites were also selected for analysis. Owing to the unavailability of neuroendocrine cells, which were believed to be the cell of origin of SCLC (Sutherland et al., 2011), normal bronchial epithelial cells were chosen as a control for these studies. Clustering analysis based on the total methylation peaks of SCLC cell lines showed that all cell lines cluster tightly together (FIG. 1). Further analysis of these methylated peaks for tumor cell line-specific peaks revealed 1223 unique tumor-specific peaks found in 4 out of 5 SCLC cell lines (≧80% of SCLC cell lines) compared with methylated peaks form normal bronchial epithelial cells. These peaks represented 676 ensemb1 gene IDs mapped to promoter regions, 323 ensemb1 gene IDs corresponding to methylated regions in the gene body and 93 ensemb1 gene IDs where the hypermethylated regions could be located downstream of genes (FIG. 4c). Individual cell lines contained between 2779 and 4485 cell line-specific methylation peaks (Table 3), numbers that were greater than those found in primary SCLCs. SCLC tumor-specific methylated regions were compared with SCLC cell line-specific methylated regions. There was a relatively small group (<20%) of SCLC cell line-specific genes found to be commonly (>6 of 18) methylated in primary SCLC tumors and vice versa (that is, ˜21% of SCLC primary tumor peaks matched with those of frequent SCLC cell line methylation; FIG. 4d). When the overlap between peaks methylated in 14/18 tumors and 4 of 5 cell lines was determined, the number of overlapped genes was 27 (FIG. 4e). The location of tumor-specific methylation peaks was mapped relative to promoters, gene bodies and locations downstream of genes (FIGS. 4a-c). The distribution patterns were similar for peaks found in ≧6/18 tumors and in cell lines, but for the most frequently methylated genes (≧14/18) the peaks tended to be more commonly localized in gene bodies and downstream (FIG. 4b). Cluster analysis of methylation peaks in normal and tumor samples is shown in FIG. 4f.

Materials and Methods

Tissue and DNA Samples.

Primary SCLC tumor tissue DNAs were obtained from patients undergoing surgery at the Nagoya University Hospital or Aichi Cancer Center, Nagoya, Japan. Pairs of human primary SCLC tumor tissue DNA and adjacent normal lung tissue DNA were obtained from Asterand (Detroit, Mich., USA), BioChain (Hayward, Calif., USA) and Cureline (South San Francisco, Calif., USA). SCLC cell lines (H1688, H1417, H1836, DMS53 and SW1271) were obtained from the ATCC (Manassas, Va., USA). The ATCC used short tandem repeat profiling for cell line identification. Normal bronchial epithelial cells (HBECs obtained from Lonza, Walkersville, Md., USA) were used as a control for the cell line analysis. All cells were cultured with Dulbecco's modified Eagle's medium/F12 with 0.5% fetal bovine serum and the bronchial epithelial growth medium bullet kit (Lonza). DNA was subjected to MIRA enrichment as described previously (Rauch and Pfeifer, 2009; Rauch and Wu et al., 2009) and subsequent microarray analysis was performed on 720k Nimblegen CpG island plus promoter arrays.

MIRA and Microarray Hybridization.

Tumor and normal tissue DNA was fragmented by sonication to ˜500 by average size as verified on agarose gels. Enrichment of the methylated double-stranded DNA fraction by MIRA was performed as described previously (Rauch and Pfeifer, 2009; Rauch and Wu et al., 2009). The labeling of amplicons, microarray hybridization and scanning were performed according to the NimbleGen (Madison, Wis., USA) protocol. NimbleGen tiling arrays were used for hybridization (Human 3×720K CpG Island Plus RefSeq Promoter Arrays). These arrays cover all UCSC Genome Browser annotated CpG islands (total of 27,728) as well as the promoters (total of 22,532) of the well-characterized RefSeq genes derived from the UCSC RefFlat files. The promoter region covered was ˜3 kb (−2440 to +610 relative to the transcription start sites). For all samples, the MIRA-enriched DNA was compared with the input DNA. All microarray data sets have been deposited into the NCBI GEO database (accession number GSE35341).

Identification and Annotation of Methylated Regions.

Analysis of the arrays was performed with R version 2.10, Perl scripts and the Bioconductor package Ringo (Toedling et al., 2007). Arrays were clustered in normal tissues, cell lines and tumor tissues using hclust and Spearman's correlation. Biological replicates were quantile-normalized and arrays were normalized by Nimblegen's recommended method, tukey's biweight. Probe ratios were smoothed for three neighboring probes before peak calling. Instead of estimating a cutoff ratio based on a hypothetical normal distribution for non-bound probes (Ringo), a quantile-based approach was chosen to estimate methylation intensities. For this aim, peaks at different quantiles were called, where four probes were above the quantile-based threshold with a distance cutoff of 300 bp. A randomized set of peaks was validated by COBRA assays (Xiong et al., 1997) for each quantile range. Thus, a quantile range of 80% was chosen as a cutoff for methylated regions (defined as hypermethylated regions). False positives and false negatives were assessed by COBRA. To investigate whether inter-sample differences had an influence on the acquired cutoff, predicted peaks were validated in different tissues by COBRA analysis.

Tumor specific regions were defined using two different stringencies. In one case, an overlap of peaks in 6 or more out of 18 tumor samples (33%) was required above the cutoff quantile threshold of 80%; the genomic regions were defined and for those regions only one out of five normal tissues was allowed to overlap with a peak called on a 56% basis, which resulted in an at least 1.5 ratio change. Overlaps were calculated using BEDtools (Quinlan et al., 2010). A more stringent analysis required an overlap of peaks in at least 14 out of 18 tumors (>77%), with the same settings as above. The obtained chromosomal positions were converted to the latest hgl9 genome build, using LiftOver from UCSC, requiring a minimum ratio of 0.9 of bases that must remap. The obtained positions where then annotated using the Bioconductor package ChlPpeakAnno and the latest ensemb1e annotation from BioMart (Sanger Institute, Cambridge, UK).

### Example 2

### Validation of Gene-Specific Methylation in SCLC Samples

Tumor-specific methylation peaks discovered by the microarray analysis described in Example 1 were further validated for several of the targets using the COBRA assay. In this example, bisulfite-converted DNA was PCR-amplified using gene-specific primers and was then digested with a restriction endonuclease, either BstUI or TaqI, which recognizes the sequences 5′-CGCG-3′ or 5′-TCGA-3′, respectively. In the COBRA assay, the cytosines in unmethylated restriction sites are converted by sodium bisulfite, amplified by PCR, and resist digestion, whereas methylated sites remain unchanged and are cleaved by these enzymes. The digested fragments visualized on agarose gels are thus indicative of methylated restriction sites in the region analyzed. An extensive validation analysis was performed by COBRA to confirm the tumor-specific methylated regions (FIG. 6). Representative examples of COBRA results are shown for the genes DMRTA2, MIR-129-2 and GALNTL1. In total, the methylation status of 11 genes (GALNTL1, MIR-10A, MIR-129-2, MIR-196A2, MIR-615, MIR-9-3, AMBRA1, HOXD10, PROX1, ZNF672 and DMRTA2) was inspected based on the various degrees of methylation obtained from the list of differentially methylated targets. Results for all the targets are presented in Table 4.

TABLE 4
Validation of frequently methylated genes by COBRA assays.

| Gene | | | | | | | | | | | | | | | | | | | |
|---|---|---|---|---|---|---|---|---|---|---|---|---|---|---|---|---|---|---|---|
| target | T1 | T2 | T3 | T4 | T5 | T6 | T7 | T8 | T9 | T11 | T12 | T13 | T14 | T15 | T16 | T17 | T18 | N1 | N3 |
| GALNTL1 | FN | + | + | | + | + | + | FN | FN | + | | | FP | | + | | | | |
| MIR10A | | | | + | + | | | + | | + | | | + | + | | + | | | |
| MIR129-2 | + | + | | + | | + | + | + | + | | + | + | FN | + | | FP | | | |
| MIR196A2 | + | + | | | + | | + | + | | + | | | | | | | | | |
| MIR615 | + | + | + | + | FN | + | + | + | + | FN | + | + | + | + | | | | | + |
| MIR9-3 | | + | + | + | + | FN | + | + | + | + | FN | + | + | + | + | FP | | | |
| AMBRA1 | | + | | + | + | | + | + | + | | + | | + | + | FN | + | + | | |
| HOXD10 | + | + | + | + | + | + | + | + | + | + | + | + | + | + | | + | + | | + |
| PROX1 | + | + | + | + | + | + | + | + | + | + | | + | + | + | | + | + | | |
| ZNF672 | + | + | + | + | + | + | + | + | + | + | + | FP | + | + | | FP | FP | | |
| DMRTA2 | + | + | + | + | + | + | + | + | + | + | + | + | + | + | | | + | | |

Tumor-specifically methylated genes were randomly selected for COBRA analysis to verify the sensitivity and specificity of our peak-calling program. DNA from various samples (top row) was bisulfite-converted, PCR-amplified with gene-specific primers and restriction digested with BstUI. This result shows high accuracy (~93%) with ~4.5% false negative (FN) and ~3% false positive (FP) hits.
The + symbol indicates that a methylation peak from the arrays was verified by COBRA.
Empty boxes mean that no methylation peak was identified by the peak-calling program and we there was no methylation found by COBRA either.

The COBRA analysis revealed that the microarray analysis described in Example 1 is highly reliable with over 93% accuracy and only ˜4% false negative and ˜3% false positive hits. To further confirm the COBRA results of the methylated genes GALNTL1 and DMRAT2, bisulfite-converted DNA was sequenced from SCLC tumor and matched normal lung samples (FIG. 7). Normal control lung DNA samples showed either no or very low levels of methylation across the CpG dinucleotides tested in contrast to SCLC tumor DNA samples, which were heavily methylated.

Discussion

Eleven methylated genomic regions, which were predicted by the array analysis, were randomly selected and validated by using bisulfite-based COBRA assays. Some of the validated genes were epigenetically altered in various other cancers (e.g. MIR-10A, MIR-129-2, MIR-196A2, HOXD10 and PROX1), but other genes have not yet been identified as methylated in any cancer type (e.g. GALNTL1, MIR-615, AMBRA1, ZNF672 and DMRTA2). Given the strong enrichment for neuronal differentiation pathways in tumor specific methylated regions in SCLC (FIG. 8), without being bound to any specific mechanism, it was possible that there was a contribution of DMRTA2 methylation to impaired homeostasis between DMRTA2 and NFIA. There was no functional evidence yet for GALNTL 1. These two targets, as well as the many other very frequently methylated genes (FIG. 5), had the potential to be used as biomarkers for SCLC.

Materials and Methods

DNA methylation analysis using sodium bisulfite-based methods. DNA was treated and purified with the EZ DNA Methylation-Gold Kit (Zymo Research, Irvine, Calif., USA). PCR primer sequences for amplification of specific gene targets in bisulfite-treated DNA are shown in Table 5.

TABLE 5
Primers for COBRA and bisulfate sequencing.

| Gene | Forward Primer | Reverse Primer |
|---|---|---|
| GALNTL1 | TGTTTTTTGTTTGGAGTGAAGAGTA | CCCAAAACCACACAACTAATTAATAA |
| MIR-10A | TTTTGTAGTTGGATGGGGAAG | TCTATCTATAATATAAAAAACCAAATC |
| MIR-129-2 | GGAGATATTTTGGGTTGAAGG | CAAATACTTTTTAAAATAAAAACTTCC |
| MIR-196A2 | TTTTATTTTTGGTTGATAAATATGA | TTTAAACCCCAAACTTAAAACAATC |
| MHZ-615 | ATTGGAGAAGGAATTTTATTTTAAT | TTCTAAAACCAAATTTTAATCTATC |
| MIR-9-3 | GAGTTTAAAAGGTAGTTGAGGGTG | ATTTAACTACTTCCAAATTCTTTATTCAAA |
| AMBRA1 | TTTTTTTTATGTGAGGGAGGTTTA | CACAAAACCAAAACCCAAACTAC |
| HOXD10 | ATTTGGAGGTTTTTAGAGTTGAGATT | CACATAACAACCAAACCAATAAAATT |
| PROX1 | GTATTTTTAGTAGGTTGAGAGGG | CTAAATCTAACAAAAACTCCAACCC |
| ZNF672 | GTGGGGTTAGTTTTAGTTATATT | CTCTTAAAACAATATTCCCCAAC |
| DMRTA2 | TTGTTTTTGATTGTGTAATGGGTAG | CCTTAACCTCAACTCCAAACTATCA |

The PCR products were analyzed by COBRA as described previously (Xiong et al., 1997). In addition, PCR products from bisulfite-converted DNA were cloned into pCR2.1-TOPO using a TOPO TA cloning kit (Invitrogen, Carlsbad, Calif., USA), and individual clones were sequenced with the M13 forward (−20) primer.

### Example 3

### Gene Expression and Methylation Status

For the SCLC cell lines SW1271, H1836 and H1688, and HBECs, Affymetrix gene expression analysis was performed and hypermethylated regions in the SCLC cell lines were compared with their associated probe expression changes. On a global level, a correlation between the tumor-specific hypermethylated regions and downregulation of associated genes could not be detected. This phenomenon has been observed in other tumor methylation studies. Some of the reasons for this lack of correlation are that (1) genes that become methylated in tumors frequently are already expressed at very low levels in corresponding normal tissues (Hahn et al., 2008; Reinert et al., 2011; Rodriguez et al., 2008; Takeshima et al., 2009), (2) methylation-independent mechanisms (such as chromatin modifications) are responsible for expression changes (Kondo et al., 2008) and (3) methylation of alternative promoters obscures such correlations (Rauch and Wu et al., 2009; Maunakea et al., 2010). Unlike the methylation patterns, the expression signals of the individual tumor cell lines were not highly correlated to each other when compared with the control cell line (as seen by principal component analysis).

Materials and Methods

Microarray Expression Analysis.

Affymetrix (Santa Clara, Calif., USA) human U133plus2.0 arrays for the three cell lines SW1271, H1836 and H1688 were processed by the robust multi-array average method implemented in the Bioconductor ‘Affy’ package, and the average log 2 intensity of each gene across all samples was calculated. The three cell lines were clustered and compared against the control cell line, HBECs. Single expression values were obtained, using the MAS 5.0 method. Proximal promoter hypermethylated and non-hypermethylated regions, defined as −2000 to +1000 by relative to transcription start sites according to the NimbleGen tilling arrays, were assigned with their respective expression probe changes of the corresponding transcript. The correlation between methylation and gene expression was based on a binary decision, linking gene promoters with differentially methylated regions with gene expression changes. A comparison with gene expression changes, where the promoter regions had a change in their methylation levels (as measured by peak detected or absent), was above the significance threshold (P-value 0.05, two-sided t-test).

### Example 4

### Functional Pathway Analysis of Methylated Genes

For the two stringencies that were defined (≧6 out of 18 tumors specifically hypermethylated and ≧14 out of 18 tumors specifically hypermethylated) as described in the Examples above, a functional annotation clustering was performed for promoter proximal tumor-specifically methylated regions and gene body-associated tumor-specifically methylated regions. For ≧6 out of 18 tumor specific promoter proximal methylated regions, two main annotation clusters could be identified, one for homeobox genes (P-value 1.6E-26, Bonferroni corrected) and one for transcription factors in general (1.0E-09; FIG. 8a). SCLC patients investigated in the study described herein, showed a strong enrichment of tumor-specific methylation at homeobox genes in promoter and gene body regions (FIGS. 9 and 10, respectively). More specifically, clusters for neuronal fate commitment (1.3E-5), neuronal differentiation (3.5E-9) and pattern specification processes (2.3E-11) showed the strongest enrichment. In comparison, hypermethylated regions in gene bodies showed similar functional enrichment clusters for homeobox genes (6.2E-26) and pattern specification processes (3.8E-11), but significantly less enrichment for neuronal fate commitment (7.0E-1) and for neuronal differentiation (1.2E-4), suggesting that the latter functional categories are more related to promoter-specific methylation (FIG. 8a).

Concerning functional enrichment for tumor-specifically hypermethylated regions for the majority of tumors (≧14 out of 18 tumors), clusters with significantly less enrichment compared with their less significant counterpart (≧6 out of 18) could only be obtained for homeobox genes (7.5E-7 for promoter regions and 2.3E-8 for gene bodies) and transcription factors (2.8E-4 for promoter regions and 3.6E-2 for gene bodies), which can be partly explained by the lower number of genes in this category. Lung development was another significantly enriched category for promoter methylation.

With regard to the cell lines, genes associated with hypermethylated regions in the five SCLC cell lines compared with the control cell line, homeobox-related functional terms and transcription factor-related terms were significantly enriched only for gene body-associated tumor peaks (4.8E-8 for homeobox genes and 3.0E-3 for transcription factors, Bonferroni corrected), but the strong enrichment for these categories observed for promoter regions in the tumor tissues was not present for the cell line models. This probably reflects a greater number and higher diversity of methylation events observed in the cell lines.

For targets methylated simultaneously in ≧14 out of 18 tumors and in ≧4 out of 5 cell lines (Table 6), enrichment was again observed in the same functional categories.

TABLE 6
Positions of tumor-specific methylation sites common between
cell lines (≧4 out of 5) and primary tumors (≧14 out of 18).

| chr | start | stop | Ensembl_ID | HGNC.symbol | Description |
|---|---|---|---|---|---|
| chr7 | 97360940 | 97362189 | ENSG00000006128 | TAC1 | tachykinin, precursor 1 |
| chr20 | 30639265 | 30639939 | ENSG00000101336 | HCK | hemopoietic cell kinase |
| | | | | | single-minded homolog |
| chr6 | 100911555 | 100911904 | ENSG00000112246 | SIM1 | 1 (Drosophila) |
| chr7 | 8474326 | 8475225 | ENSG00000122584 | NXPH1 | neurexophilin 1 |
| chr7 | 8483076 | 8483825 | ENSG00000122584 | NXPH1 | neurexophilin 1 |
| chr2 | 176969205 | 176970504 | ENSG00000128713 | HOXD11 | homeobox D11 |
| chr1 | 197879928 | 197880227 | ENSG00000143355 | LHX9 | LIM homeobox 9 |
| | | | | | T-cell acute lymphocytic |
| chr1 | 47694864 | 47695213 | ENSG00000162367 | TAL1 | leukemia 1 |
| | | | | | LIM homeobox |
| | | | | | transcription factor 1, |
| chr1 | 165323327 | 165323876 | ENSG00000162761 | LMX1A | alpha |
| chr3 | 27765097 | 27765996 | ENSG00000163508 | EOMES/TBR2 | eomesodermin |
| chr4 | 85402627 | 85403376 | ENSG00000163623 | NKX6-1 | NK6 homeobox 1 |
| | | | | | heart and neural crest |
| chr4 | 174448276 | 174448725 | ENSG00000164107 | HAND2 | derivatives expressed 2 |
| | | | | | zinc finger and BTB |
| chr9 | 129566330 | 129566679 | ENSG00000169155 | ZBTB43 | domain containing 43 |
| | | | | | vestigial like 2 |
| chr6 | 117584408 | 117584857 | ENSG00000170162 | VGLL2 | (Drosophila) |
| chr2 | 177027205 | 177027529 | ENSG00000170166 | HOXD4 | homeobox D4 |
| chr3 | 183274057 | 183274331 | ENSG00000172578 | KLHL6 | kelch-like 6 (Drosophila) |
| | | | | | regulated endocrine- |
| | | | | | specific protein 18 |
| chr2 | 220196257 | 220197006 | ENSG00000182698 | RESP18 | homolog (rat) |
| | | | | | SRY (sex determining |
| chr13 | 112719950 | 112720174 | ENSG00000182968 | SOX1 | region Y)-box 1 |
| chr5 | 172660795 | 172660844 | ENSG00000183072 | NKX2-5 | NK2 homeobox 5 |
| | | | | | chromosome 14 open |
| chr14 | 29243250 | 29243899 | ENSG00000186960 | C14orf23 | reading frame 23 |
| chr19 | 9608951 | 9609250 | ENSG00000198028 | ZNF560 | zinc finger protein 560 |
| chr6 | 27107272 | 27107346 | ENSG00000198339 | HIST1H4I | histone cluster 1.H41 |
| chr8 | 9756191 | 9756540 | ENSG00000208010 | MIR124-1 | microRNA 124-1 |
| chr4 | 122685401 | 122685475 | ENSG00000226757 | | Uncharacterized protein |
| | | | | | interferon, alpha 12, |
| chr9 | 21402751 | 21403100 | ENSG00000235108 | IFNA12P | pseudogene |
| chr2 | 177004205 | 177004604 | ENSG00000237380 | | |
| chr7 | 129422815 | 129423514 | ENSG00000242078 | | |

Notably, this group of genes contained a number of genes involved in neuronal or neuroendocrine differentiation, such as EOMES/TBR2, the gene TAC1, which encodes the neuropeptide substance P, and RESP18, encoding a neuroendocrine-specific protein.

Discussion

Gene annotation analysis of tumor-specific promoter methylated targets revealed a substantial subgroup of genes that are specific for neuronal fate commitment, neuronal differentiation and pattern specification processes, along with homeobox and other transcription factors. In comparison, hypermethylated regions in gene bodies showed similar functional enrichment clusters for homeobox genes and pattern specification processes, but significantly less enrichment for neuronal fate commitment and for neuronal differentiation, suggesting that the latter functional categories are more specific for promoter-specific methylation. This striking tendency for methylation of neuronal-specific genes may suggest an essential role of this event in SCLC tumor initiation.

Methylation of surrounding proximal promoters is often tightly associated with transcriptional silencing, whereas gene body methylation seems to be associated with transcriptional activation (Rauch and Wu et al., 2009; Suzuki and Bird, 2008). Loss of expression of genes, which are methylated in their proximal promoters, could lead to SCLC tumor initiation. Further studies in this direction will be required to establish experimental evidence. What is not known at present is whether these genes are unmethylated and expressed in pulmonary neuroendocrine cells and their precursors, the likely cells of origin for SCLC. This specific cell type is currently not available for analysis. This issue does indeed apply to almost all DNA methylation studies done in human cancer to date. The exact cell of origin, the cell from which the tumor initiates, is often not known, or these cells are not available in sufficient quantities. Therefore—at least theoretically—all DNA methylation ‘changes’ found in tumor DNA may already preexist in the cell of origin. However, methylation of genes that promote the differentiation of neuroendocrine cells would be unlikely to occur in such cells as that would interfere with their normal differentiated state.

The SCLC patients investigated herein showed a strong enrichment of tumor-specific methylation at homeobox genes (FIGS. 9 and 10). Homeobox genes and other transcriptional regulators are important for developmental processes, having important roles in cellular identity, growth, differentiation and cellular interactions within the tissue environment. Given the results of the study described in this Example and the other Examples herein, disruptions in the early phase of these processes could increase the probability of the cell to become malignant, as this would lead to a pool of cells, which are aberrantly kept in a proliferation loop without a decision toward a specific cell fate. It is thought that the cells of origin for SCLC are neuroendocrine cells, as shown in mice (Sutherland et al., 2001; Park et al., 2011). Given the fact that many of the tumor-specifically methylated targets that were identified are important for cell fate decisions toward the neuronal lineage, without being bound to any specific mechanism, it was possible that one way of shifting the balance toward the emergence of SCLC would be through the repression of key factors critical for differentiation of neuroendocrine cells. One potential way of aberrant shutdown of these critical factors would be by promoter-targeted methylation. Being freed of their normal developmental program by the absence or reduction of cell fate specification factors, some of these cells could acquire additional malignant traits, according to the ‘hallmark’ model defined by Hanahan and Weinberg (Hanahan et al., 2011). This means that the observed hypermethylated regions are more probable to arise at an early stage of perturbed differentiation rather than during the later stages of tumorigenesis. Concerning other tumor-driving aberrant methylation events, which might increase the tumorigenic potential, it is interesting to note that promoter-specific methylation was rarely detected close to known tumor suppressor genes. Exceptions were tumor-specific methylation of TCF21 (Smith et al., 2006), which was detected downstream of the gene in the tumors but overlapping with the TSS in the cell lines and methylation of the promoter of the RASSFIA gene confirming earlier gene-specific studies (Burbee et al., 2001; Dammann et al., 2001).

Materials and Methods

Functional Annotation Analysis.

Gene ontology analysis was performed using DAVID functional annotation tools with Biological Process FAT and Molecular Function FAT data sets (Huang et al., Nat. Protoc., 2009; Huang et al., Nucleic Acids Res., 2009). The enriched gene ontology terms were reported as clusters to reduce redundancy. The P-value for each cluster is the geometric mean of the P-values for all the GO categories in the cluster. The gene list in each cluster contains the unique genes pooled from the genes in all the GO categories in the cluster. Functional terms were clustered by using a Multiple Linkage Threshold of 0.5 and Bonferroni corrected P-values.

### Example 6

### Sequence Motif Discovery

Next, the de novo motif discovery algorithm HOMER (Heinz et al., 2010) was used to search for sequence patterns that were associated with regions that were specifically methylated in SCLC tumor samples for at least 33% of the tumors. A set of nonredundant sequence motifs were identified that were highly enriched in comparison with all non-tumor-specifically methylated regions on the array. Transcription factors, which fell into this category, were REST/NRSF (2.5E-16), ZNF423 (3.0E-13), HAND1 (1.44E-10) and NEUROD1 (2.3E-10; FIG. 8b). Examples of methylated NEUROD1 targets are shown in FIG. 11. The majority of the sequence motifs identified in methylated regions were enriched within the proximal promoter regions of known genes. The highest enrichment was based on redundant sequence structures and for those that were not, a stringent alignment with matching transcription factor-binding sites and a low number of occurrences in the background set was demanded, which contained all possible methylation sites. REST, ZNF423, HAND1 and NEUROD1 contained nonredundant sequences, a maximal mismatch of 2 by to the identified de novo motif and were selectively enriched in the target sequence set. As such, the identified motifs might not be representative for the whole tumor-specific target set but shed light on sub-regulatory networks with a possibly major impact on the phenotype of SCLC. For example, NEUROD1- and HAND 1-binding sites were found in methylated targets representing genes involved in neuronal cell fate commitment such as GDNF, NKX2-2, NKX6-1, EVX1 and SIM2 (see Table 7).

TABLE 7
Genes involved in neuronal fate commitment, correlated
with hyper methylation in at least 33% of the tumors.*

| Ensembl ID | HGNC symbol | Description |
|---|---|---|
| ENSG00000009709 | PAX7 | paired box 7 [Source: HGNC Symbol;Acc: 8621] |
| ENSG00000129514 | FOXA1 | forkhead box A1 [Source: HGNC Symbol;Acc: 5021] |
| ENSG00000135903 | PAX3 | paired box 3 [Source: HGNC Symbol;Acc: 8617] |
| ENSG00000128710 | HOXD10 | homeobox D10 [Source: HGNC Symbol;Acc: 5133] |
| ENSG00000125820 | NKX2-2 | NK2 homeobox 2 [Source: HGNC Symbol;Acc: 7835] |
| ENSG00000169840 | GSX1 | GS homeobox 1 [Source: HGNC Symbol;Acc: 20374] |
| ENSG00000106038 | EVX1 | even-skipped homeobox 1 [Source: HGNC Symbol;Acc: 3506] |
| ENSG00000205927 | OLIG2 | oligodendrocyte lineage transcription factor 2 [Source: HGNC |
| | | Symbol;Acc: 9398] |
| ENSG00000180818 | HOXC10 | homeobox C10 [Source: HGNC Symbol;Acc: 5122] |
| ENSG00000163623 | NKX6-1 | NK6 homeobox 1 [Source: HGNC Symbol;Acc: 7839] |
| ENSG00000136352 | NKX2-1 | NK2 homeobox 1 [Source: HGNC Symbol;Acc: 11825] |

*Gene IDs were derived by DAVID gene ontology analysis using all gene IDs related to hypermethylation in at least 33% of the tumors and selecting genes belonging to GO:0048663.

Methylation of these binding sites suggests a model in which these transacting factors were lost during tumorigenesis rendering their target sites susceptible to methylation. To analyze this scenario further, the NEUROD 1 transcription factor was studied further. Expression of NEUROD1 proved to be undetectable by a sensitive reverse transcription—PCR assay (FIG. 12) in the four SCLC cell lines tested and it was expressed at very low levels in human bronchial epithelial cells. In SCLC cell lines and, importantly, also in primary SCLC tumors, the promoter of NEUROD1 was heavily methylated (FIGS. 13A and B) consistent with a possible lack of expression. In addition, increased methylation was found at the promoters of HAND1 and REST in SCLC cell lines and in primary tumors (FIGS. 13A and B).

Materials and Methods

De Novo Motif Prediction.

Motif analysis was performed by HOMER, a program developed by Heinz et al., 2010. More specifically, the discovery was performed using a comparative algorithm similar to those previously described by Linhart et al, 2008. Briefly, sequences were divided into target and background sets for each application of the algorithm (choice of target and background sequences are noted below). Background sequences were then selectively weighted to equalize the distributions of CpG content in target and background sequences to avoid comparing sequences of different general sequence content. Motifs of length 8-30 by were identified separately by first exhaustively screening all possible oligos for enrichment in the target set compared with the background set by assessing the number of target and background sequences containing each oligo and then using the cumulative hypergeometric distribution to score enrichment. Up to two mismatches were allowed in each oligonucleotide sequence to increase the sensitivity of the method. The top 200 oligonucleotides of each length with the best enrichment scores were then converted into basic probability matrices for further optimization. HOMER then generates motifs comprised of a position-weight matrix and detection threshold by empirically adjusting motif parameters to maximize the enrichment of motif instances in target sequences versus background sequences using the cumulative hypergeometric distribution as a scoring function. Probability matrix optimization follows a local hill-climbing approach that weights the contributions of individual oligos recognized by the motif to improve enrichment, while optimization of motif detection thresholds were performed by exhaustively screening degeneracy levels for maximal enrichment during each iteration of the algorithm. Once a motif is optimized, the individual oligos recognized by the motif are removed from the data set to facilitate the identification of additional motifs. Sequence logos were generated using WebLOGO (Crooks et al., 2004). Motifs obtained from Jasper and TRANSFAC for which no high-throughput data exists were discarded for this analysis. Only those motifs with the highest alignments to known transcription factors, nonredundant matrixes and non-repetitive sequences were chosen for further analysis.

Transfection, Reverse Transcription and Quantitative Real-Time PCR.

The DMS53 SCLC line was transfected with a NEUROD1 expression plasmid (2 mg) at ˜60% confluence in 35-mm dishes with FuGENE HD (Roche Applied Science, Indianapolis, Ind., USA) in serum-free medium according to the manufacturer's recommendations. The cells were cultured for an additional 48 h for analysis of NEUROD1 expression. Total RNA was isolated from HBECs, all five SCLC cell lines and from DMS53 cells overexpressing NEUROD1 using the RNeasy Mini Kit (Qiagen). cDNA was prepared using the iScript cDNA synthesis kit (Bio-Rad; Hercules, Calif., USA). Quantitative PCR was performed to assess expression of NEUROD1 and 18S RNA using NEUROD1 primers (forward, 5′-GTTCTCAGGACGAGGAGCAC-3′ and reverse 5′-CTTGGGCTTTTGATCGTCAT-3′) and 18S primers (forward 5′-GTAACCC GTTGAACCCCATT-3′ and reverse 5′-C CAT C CAATC GGTAGTAGC G-3′). Realtime PCR was performed using iQ SYBR Green Supermix and the iCycler real-time PCR detection system (Bio-Rad). Amplicon expression in each sample was normalized to 18S RNA.

### REFERENCES

The references, patents and published patent applications listed below, and all references cited in the specification above are hereby incorporated by reference in their entireties, as if fully set forth herein.
Burbee D G, Forgacs E, Zochbauer-Muller S, Shivakumar L, Fong K, Gao B et al. Epigenetic inactivation of RASSF1A in lung and breast cancers and malignant phenotype suppression. J Natl Cancer Inst 2001; 93: 691-699.
Clark S. J., Harrison, J., Paul, C. L., Frommer, M., (1994): ‘High sensitivity mapping of methylated cytosines’ Nucleic Acids Res. 22:2990-2997.
Costello, J. F., M. C. Fruhwald, et al. (2000). “Aberrant CpG-island methylation has non-random and tumour-type-specific patterns.” Nat Genet. 24(2): 132-8.
Coulson J M. Transcriptional regulation: cancer, neurons and the REST. Curr Biol 2005; 15: R665-R668.
Coulson J M, Edgson J L, Woll P J, Quinn J P. A splice variant of the neuron-restrictive silencer factor repressor is expressed in small cell lung cancer: a potential role in derepression of neuroendocrine genes and a useful clinical marker. Cancer Res 2000; 60: 1840-1844.
Crooks, G. E., et al. WebLogo: A Sequence Logo Generator, Genome Research, 14:1188-1190, (2004), http://weblogo.berkeley.edu/.
Dammann R, Li C, Yoon J H, Chin P L, Bates S, Pfeifer G P. Epigenetic inactivation of a RAS association domain family protein from the lung tumour suppressor locus 3p21.3. Nat Genet. 2000; 25: 315-319.
Dammann R, Takahashi T, Pfeifer G P. The CpG island of the novel tumor suppressor gene RASSF1A is intensely methylated in primary small cell lung carcinomas. Oncogene 2001; 20: 3563-3567.
De Hoon, M., Imoto, S., Nolan, J., Miyano, S., Open Source Clustering Software. Bioinformatics, 20 (9): 1453-1454 (2004), http://bonsai.hgc.jp/˜mdehoon/software/cluster/software.htm.
Fischer B, Marinov M, Arcaro A. Targeting receptor tyrosine kinase signaling in small cell lung cancer (SCLC): what have we learned so far? Cancer Treat Rev 2007; 33: 391-406.
Fraga M F, Ballestar E, Montoya G, et al. The affinity of different MBD proteins for a specific methylated locus depends on their intrinsic binding properties. Nucleic Acids Res 2003; 31:1765-1774.
Govindan R, Page N, Morgensztern D, Read W, Tierney R, Vlahiotis A et al. Changing epidemiology of small-cell lung cancer in the United States over the last 30 years: analysis of the surveillance, epidemiologic, and end results database. J Clin Oncol 2006; 24: 4539-4544.
Hahn M A, Hahn T, Lee D H, Esworthy R S, Kim B W, Riggs A D et al. Methylation of polycomb target genes in intestinal cancer is mediated by inflammation. Cancer Res 2008; 68: 10280-10289.
Hanahan D, Weinberg R A. Hallmarks of cancer: the next generation. Cell 2011; 144: 646-674.
HERMAN J G, GRAFF J R, MYOHANEN S, NELKIN B D, BAYLIN S B (1996): “Methylation-specific PCR: a novel PCR assay for methylation status of CpG islands.” Proc. Natl. Acad. Sci. U.S.A. 93:9821-9826
Heinz S, Benner C, Spann N, Bertolino E, Lin Y C, Laslo P et al. Simple combinations of lineage-determining transcription factors prime cis-regulatory elements required for macrophage and B cell identities. Mol Cell 2010; 38: 576-589.
Holzel M, Huang S, Koster J, Ora I, Lakeman A, Caron H et al. NF1 is a tumor suppressor in neuroblastoma that determines retinoic acid response and disease outcome. Cell 2010; 142: 218-229.
Huang S, Laoukili J, Epping M T, Koster J, Holzel M, Westerman B A et al. ZNF423 is critically required for retinoic acid-induced differentiation and is a marker of neuroblastoma outcome. Cancer Cell 2009; 15: 328-340.
Huang da W, Sherman B T, Lempicki R A. Systematic and integrative analysis of large gene lists using DAVID bioinformatics resources. Nat Protoc 2009; 4: 44-57.
Huang da W, Sherman B T, Lempicki R A. Bioinformatics enrichment tools: paths toward the comprehensive functional analysis of large gene lists. Nucleic Acids Res 2009; 37: 1-13.
Johnson B E, Ihde D C, Makuch R W, Gazdar A F, Carney D N, Oie H et al. myc family oncogene amplification in tumor cell lines established from small cell lung cancer patients and its relationship to clinical status and course. J Clin Invest 1987; 79: 1629-1634.
Kalari S, Pfeifer G P. Identification of driver and passenger DNA methylation in cancer by epigenomic analysis. Adv Genet. 2010; 70: 277-308.
Kondo Y, Shen L, Cheng A S, Ahmed S, Boumber Y, Charo C et al. Gene silencing in cancer by histone H3 lysine 27 trimethylation independent of promoter DNA methylation. Nat Genet. 2008; 40: 741-750.
Kreisler A, Strissel P L, Stick R, Neumann S B, Schumacher U, Becker C M. Regulation of the NRSF/REST gene by methylation and CREB affects the cellular phenotype of small-cell lung cancer. Oncogene 2010; 29: 5828-5838.
Laird P W. The power and the promise of DNA methylation markers. Nat Rev Cancer 2003; 3: 253-266.
Lee N H, Haas B J, Letwin N E, Frank B C, Luu T V, Sun Q et al. Cross-talk of expression quantitative trait loci within 2 interacting blood pressure quantitative trait loci. Hypertension 2007; 50: 1126-1133.
Lerman M I, Minna J D. The 630-kb lung cancer homozygous deletion region on human chromosome 3p21.3: identification and evaluation of the resident candidate tumor suppressor genes. The International Lung Cancer Chromosome 3p21.3 Tumor Suppressor Gene Consortium. Cancer Res 2000; 60: 6116-6133.
Linhart C, Halperin Y, Shamir R. Transcription factor and microRNA motif discovery: the Amadeus platform and a compendium of metazoan target sets. Genome Res 2008; 18: 1180-1189.
Little C D, Nau M M, Carney D N, Gazdar A F, Minna J D. Amplification and expression of the c-myc oncogene in human lung cancer cell lines. Nature 1983; 306: 194-196.
Majumder S. REST in good times and bad: roles in tumor suppressor and oncogenic activities. Cell Cycle 2006; 5: 1929-1935.
Maunakea A K, Nagarajan R P, Bilenky M, Ballinger T J, D'Souza C, Fouse S D et al. Conserved role of intragenic DNA methylation in regulating alternative promoters. Nature 2010; 466: 253-257.
Modi S, Kubo A, Oie H, Coxon A B, Rehmatulla A, Kaye F J. Protein expression of the RB-related gene family and SV40 large T antigen in mesothelioma and lung cancer. Oncogene 2000; 19: 4632-4639.
Neptune E R, Podowski M, Calvi C, Cho J H, Garcia J G, Tuder R et al. Targeted disruption of NeuroD, a proneural basic helix-loop-helix factor, impairs distal lung formation and neuroendocrine morphology in the neonatal lung. J Biol Chem 2008; 283: 21160-21169.
Park K S, Liang M C, Raiser D M, Zamponi R, Roach R R, Curtis S J et al. Characterization of the cell of origin for small cell lung cancer. Cell Cycle 2011; 10: 2806-2815.
PFEIFER G P, STEIGERWALD S D, MUELLER PR, WOLD B, RIGGS A D (1989). “Genomic sequencing and methylation analysis by ligation mediated PCR.” Science 246(4931):810-813.
Pfeifer et al. (2007) “Methylated-CpG island recovery assay-assiated microassays for cancer diagnosis” Expert Opin. Med. Diagn. 1(1):1-10.
Piper M, Barry G, Hawkins J, Mason S, Lindwall C, Little E et al. NFIA controls telencephalic progenitor cell differentiation through repression of the Notch effector Hesl. J Neurosci 2010; 30: 9127-9139.
Quinlan A R, Hall I M. BEDToo1s: a flexible suite of utilities for comparing genomic features. Bioinformatics 2010; 26: 841-842.
Qureshi I A, Gokhan S, Mehler M F. REST and CoREST are transcriptional and epigenetic regulators of seminal neural fate decisions. Cell Cycle 2010; 9: 4477-4486.
Rauch, T. and G. P. Pfeifer (2005). “Methylated-CpG island recovery assay: a new technique for the rapid detection of methylated-CpG islands in cancer.” Lab Invest 85(9): 1172-80.
Rauch T A, Pfeifer G P. DNA methylation profiling using the methylated-CpG island recovery assay (MIRA). Methods 2010; 52: 213-217.
Rauch T, Wang Z, Zhang X, Zhong X, Wu X, Lau S K et al. Homeobox gene methylation in lung cancer studied by genome-wide analysis with a microarraybased methylated CpG island recovery assay. Proc Natl Acad Sci USA 2007; 104: 5527-5532.
Rauch T A, Wu X, Zhong X, Riggs A D, Pfeifer G P. A human B cell methylome at 100-base pair resolution. Proc Natl Acad Sci USA 2009; 106: 671-678.
Rauch T A, Zhong X, Wu X, Wang M, Kernstine K H, Wang Z et al. High-resolution mapping of DNA hypermethylation and hypomethylation in lung cancer. Proc Natl Acad Sci USA 2008; 105: 252-257.
Reinert T, Modin C, Castano F M, Lamy P, Wojdacz T K, Hansen L L et al. Comprehensive genome methylation analysis in bladder cancer: identification and validation of novel methylated genes and application of these as urinary tumor markers. Clin Cancer Res 2011; 17: 5582-5592.
Rodriguez J, Munoz M, Vives L, Frangou C G, Groudine M, Peinado M A. Bivalent domains enforce transcriptional memory of DNA methylated genes in cancer cells. Proc Natl Acad Sci USA 2008; 105: 19809-19814.
Saldanha, A. Java Treeview-extensible visualization of microarray data. Bioinformatics. 20 (18); 3246-3248 (2004) http://jtreeview.sourceforge.net.
Sattler M, Salgia R. Molecular and cellular biology of small cell lung cancer. Semin Oncol 2003; 30: 57-71.
Sato M, Shames D S, Gazdar A F, Minna J D. A translational view of the molecular pathogenesis of lung cancer. J Thorac Oncol 2007; 2: 327-343.
Sekido Y, Fong K M, Minna J D. Molecular genetics of lung cancer. Annu Rev Med 2003; 54: 73-87.
Singer J, Roberts-Ems J, Riggs A D. (1979) Methylation of mouse liver DNA studied by means of the restriction enzymes msp I and hpa II. Science 203:1019-1021.
Smith L T, Lin M, Brena R M, Lang J C, Schuller D E, Otterson G A et al. Epigenetic regulation of the tumor suppressor gene TCF21 on 6q23-q24 in lung and head and neck cancer. Proc Natl Acad Sci USA 2006; 103: 982-987.
Stadler M B, Murr R, Burger L, Ivanek R, Lienert F, Scholer A et al. DNA-binding factors shape the mouse methylome at distal regulatory regions. Nature 2011; 480: 490-495.
Sunaga N, Miyajima K, Suzuki M, Sato M, White M A, Ramirez R D et al. Different roles for caveolin-1 in the development of non-small cell lung cancer versus small cell lung cancer. Cancer Res 2004; 64: 4277-4285.
Sutherland K D, Proost N, Brouns I, Adriaensen D, Song J Y, Berns A. Cell of origin of small cell lung cancer: inactivation of Trp53 and rb1 in distinct cell types of adult mouse lung. Cancer Cell 2011; 19: 754-764.
Suzuki M M, Bird A. DNA methylation landscapes: provocative insights from epigenomics. Nat Rev Genet. 2008; 9: 465-476.
Takahashi T, Obata Y, Sekido Y, Hida T, Ueda R, Watanabe H et al. Expression and amplification of myc gene family in small cell lung cancer and its relation to biological characteristics. Cancer Res 1989; 49: 2683-2688.
Takeshima H, Yamashita S, Shimazu T, Niwa T, Ushijima T. The presence of RNA polymerase II, active or stalled, predicts epigenetic fate of promoter CpG islands. Genome Res 2009; 19: 1974-1982.
Toedling J, Skylar 0, Krueger T, Fischer J J, Sperling S, Huber W. Ringo—an R/Bioconductor package for analyzing ChIP-chip readouts. BMC Bioinformatics 2007; 8: 221.
Tommasi S, Karm D L, Wu X, Yen Y, Pfeifer G P. Methylation of homeobox genes is a frequent and early epigenetic event in breast cancer. Breast Cancer Res 2009; 11: R14. Rauch T A, Pfeifer G P. The MIRA method for DNA methylation analysis. Methods Mol Biol 2009; 507: 65-75.
Ushijima T. Detection and interpretation of altered methylation patterns in cancer cells. Nat Rev Cancer 2005; 5: 223-231.
Warming S, Rachel R A, Jenkins N A, Copeland N G. Zfp423 is required for normal cerebellar development. Mol Cell Biol 2006; 26: 6913-6922.
Wistuba I I, Gazdar A F, Minna J D. Molecular genetics of small cell lung carcinoma. Semin Oncol 2001; 28: 3-13.
Wu X, Rauch T A, Zhong X, Bennett W P, Latif F, Krex D et al. CpG island hypermethylation in human astrocytomas. Cancer Res 2010; 70: 2718-2727.
Xiong Z, Laird P W. COBRA: a sensitive and quantitative DNA methylation assay. Nucleic Acids Res 1997; 25: 2532-2534.

What is claimed is:

1. A method of diagnosing small cell lung cancer (SCLC) in a subject comprising:
measuring methylation levels of one or a combination of DNA biomarkers in a test sample of the subject;
comparing the methylation levels of the one or the combination of DNA biomarkers with the methylation levels of a corresponding one or a combination of DNA biomarkers in a normal sample or standard sample; and
predicting that an increase in the methylation levels of the test sample in relation to that of the normal sample or standard sample indicates that the subject is likely to have SCLC.

2. The method of claim 1, wherein the test sample is selected from the group consisting of lung tissue, sputum, and blood serum.

3. The method of claim 1, wherein the DNA biomarker is one or more genes listed in FIG. 5.

4. The method of claim 1, wherein the DNA biomarker is one or more genes selected from the group consisting of GALNTL1, MIR-10A, MIR-129-2, MIR-196A2, MIR-615, MIR-9-3, AMBRA1, HOXD10, PROX1, ZNF672, and DMRTA2.

5. The method of claim 1, wherein the DNA biomarker is one or more genes listed in Table 6.

6. The method of claim 1, wherein the methylation levels are measured by a methylated-CpG island recovery assay (MIRA), bisulfite sequencing, a combined bisulfite-restriction analysis (COBRA), or a methylation-specific PCR (MSP).

7. The method of claim 6, wherein the methylation levels of a combination of DNA biomarkers are measured by a MIRA-assisted microarray analysis.

8. The method of claim 1, wherein the DNA biomarker comprises a highly enriched sequence motif that is a transcription factor binding site.

9. The method of claim 8, wherein the transcription factor is selected from the group consisting of REST, ZNF423, HAND1, and NEUROD1.

10. The method of claim 9, wherein:
when the transcription factor is REST, the highly enriched sequence motif comprises a nucleotide sequence of SEQ ID NO: 1 [X¹⁻T-G-X²-X³-C-A-X⁴⁻G-G-T-G-C-T-G-A, wherein X¹ can be either C or G, X² can be either T or A, X³ can be either A or C, and X⁴ can be either A or T];
when the transcription factor is ZNF423, the highly enriched sequence motif comprises a nucleotide sequence of SEQ ID NO: 2 [G-A-A-C-C-C-T-G-C-G-G-G-T-C];
when the DNA biomarker is HAND1, the highly enriched sequence motif comprises a nucleotide sequence of SEQ ID NO: 3 [C-C-A-G-A-C-C-G-C-A-G-A-A-A]; and
when the DNA biomarker is NEUROD1, the highly enriched sequence motif comprises a nucleotide sequence of SEQ ID NO: 4 [C-A-G-A-T-T-G-C-T-A].

11. The method of claim 8, wherein the highly enriched sequence motif is determined using a de novo motif discovery algorithm.

12. The method of claim 1, wherein the increase in the methylation levels are at least a frequency of greater than about 77% of SCLC tumors.

13. A method of diagnosing small cell lung cancer (SCLC) in a subject comprising:
1) hybridizing a methylated regions of a test sample of a subject to a DNA microarray comprising one or a combination of DNA biomarkers;
2) comparing the hybridized methylated regions of the methylated regions from the genome DNA with the hybridization of the corresponding methylated regions of a normal sample or standard sample genome DNA; and
3) predicting that an increase in the methylated regions of the genome DNA hybridizing to the DNA biomarker relative to the methylated regions of the normal sample or standard sample genome DNA hybridizing to the one or a combination of DNA biomarkers indicates that the subject is likely to have SCLC.

14. The method of claim 13, further comprising one or more of the following steps:
1) obtaining a test sample from a subject;
2) obtaining a genome DNA from the test sample from the subject;
3) obtaining methylated regions from the genome DNA;

15. The method of claim 13, wherein the test sample is selected from the group consisting of lung tissue, sputum, and blood serum.

16. The method of claim 13, wherein the DNA biomarker is one or more genes listed in FIG. 5.

17. The method of claim 13, wherein the DNA biomarker is one or more genes listed in Table 6.

18. The method of claim 13, wherein the DNA biomarker is one or more genes selected from the group consisting of GALNTL1, MIR-10A, MIR-129-2, MIR-196A2, MIR-615, MIR-9-3, AMBRA1, HOXD10, PROX1, ZNF672 and DMRTA2.

19. The method of claim 13 wherein the methylation levels are measured by a methylated-CpG island recovery assay (MIRA), by bisulfite sequencing, by a combined bisulfite-restriction analysis (COBRA), or by a methylation-specific PCR (MSP).

20. The method of claim 19, wherein the methylation levels of the one or a combination of DNA biomarkers are measured by a MIRA-assisted microarray analysis.

21. A method of treating a subject for SCLC, the method comprising:
measuring methylation levels of one or a combination of DNA biomarkers in a test sample of the subject;
comparing the methylation levels of the one or the combination of DNA biomarkers with the methylation levels of a corresponding one or a combination of DNA biomarkers in a normal sample or standard sample; and
administering a therapeutically effective amount of a chemotherapy to the subject when there is an increase in the methylation levels of the test sample in relation to that of the normal sample or standard sample.

22. The method of claim 21, wherein the subject is suffering from SCLC.

23. A method of determining the success of treating a subject for SCLC by monitoring the level of a biomarker measured by a method of claim 1 over a time period following the treatment using tissue, biopsies, blood or serum of the subject.
